(19) United States
(12) Patent Application Publication
(10) Pub. No.: US 2013/0000644 A1
(43) Pub. Date: Jan. 3, 2013

(54) SYSTEMS AND METHODS FOR PROVIDING VENTILATION BASED ON PATIENT NEED

(75) Inventor: Ronald Thiessen, Mission (CA)

(73) Assignee: Nellcor Puritan Bennett LLC, Boulder, CO (US)

(21) Appl. No.: 13/173,987

(22) Filed: Jun. 30, 2011

(51) Int. Cl. A61M 16/00

(52) U.S. Cl. 128/204.23; 128/204.21; 128/204.18

(57) ABSTRACT

A ventilation system includes a controller and an alarm module in communication with the controller. The alarm module includes an alarm and an alarm emitter indicating activation of the alarm, wherein the alarm may be one or more of the following alarms: low pressure, low volume, low respiration rate, low minute volume, disconnect condition, and apnea. When the controller is in a first mode setting, the alarm emitter is activated in response to a triggering event, and when the controller is in a second mode setting, the alarm emitter is not activated in response to the triggering event. The ventilation system may include a breathing circuit, an airflow generator for delivering a ventilation airflow to the breathing circuit, and a sensor for sensing an increase in at least one of an air flow or an air pressure within the breathing circuit, thus triggering airflow.

### INTRODUCTION

Traditionally, individuals requiring chronic, “home care” or “extended care” ventilation have required tracheostomy tubes as the interface between a ventilator and the individual. In recent years, “non-invasive ventilation” has increased in popularity and is available on many critical care and home care ventilators. One type of non-invasive ventilation is referred to as “mouthpiece ventilation” (MPV), and is a common means by which a patient population is ventilated on an as-needed (or on-demand) basis, especially during daytime use of a ventilator. When an individual is ventilated via mouthpiece, the connection between the individual and the ventilator is intermittent and only occurs when the individual engages with the patient interface to initiate a breath. No exhalation valve, PEEP valve, or exhalation limb is required as the individual simply removes their mouth from mouthpiece to exhale naturally. There are limitations, however, in using existing ventilators for MPV.

Individuals using MPV with existing ventilators do so by tolerating current limitations of existing equipment on the market. As such, the settings are not ideal, and nuisance alarms are often triggered because the ventilator interprets the patient's on-demand breathing (and frequent disconnection from the interface), as incidents that require alarms. In short, the ventilator is not programmed to recognize this particular type of ventilation. Additionally, in ventilators where certain parameters may be adjusted, the patient may initiate a breath that meets, e.g., a respiration rate requirement, but the ventilator may deliver too much or too little breathing gas. This provides a patient with an unsatisfactory breath, which may cause more problems or discomfort.

Additionally, ventilators use negative pressure changes within the breathing circuit or, in some cases, volumetric flow out of the ventilator, in order to trigger breaths. Patients who are new to the use of a ventilator may have a difficult time producing the negative pressure (i.e., suction) required to trigger a breath delivery by the ventilator. For patients learning how to employ MPV, a more simplified method of triggering a breath is needed to increase usability.

### SUMMARY

In one aspect, the technology relates to a ventilation system including: a controller having a first mode setting and a second mode setting; an alarm module in communication with the controller, the alarm module comprising an alarm and an alarm emitter indicating activation of the alarm, wherein the alarm is selected from the group consisting of a low pressure alarm, a low volume alarm, a low respiration rate alarm, a low minute volume alarm, a disconnect alarm, and an apnea alarm; wherein when the controller is in the first mode setting, the alarm emitter is activated in response to a triggering event, and wherein when the controller is in the second mode setting, the alarm emitter is not activated in response to the triggering event.

In another aspect, the technology relates to a method of controlling an alarm state in a ventilation system having a controller, a power module, a sensor module, and an alarm emitter activated by at least one of a low pressure signal and a low volume signal, the method including the step of: programming the ventilation system to include an on-demand ventilation mode, wherein in the on-demand ventilation mode, the alarm emitter is not activated.

In another aspect the technology relates to a ventilation system including: a breathing circuit; an airflow generator for delivering a ventilation airflow to the breathing circuit; and a sensor for sensing at least one of an air flow or air pressure within the breathing circuit, wherein the ventilation airflow is delivered to the breathing circuit upon an increase in at least one of the air flow and the air pressure.

### BRIEF DESCRIPTION OF THE DRAWINGS

There are shown in the drawings, embodiments which are presently preferred, it being understood, however, that the technology is not limited to the precise arrangements and instrumentalities shown.

FIG. 1 depicts a breathing assistance system.

FIG. 2 depicts a method of identifying and processing triggering events in a breathing assistance system.

FIG. 3A depicts a pressure waveform associated with a prior art breathing assistance system.

FIG. 3B depicts a pressure waveform associated with a breathing assistance system.

### DETAILED DESCRIPTION

As used herein, the term “gas” may refer to any one or more gases and/or vaporized substances suitable to be delivered to and/or from a patient via one or more breathing orifices (e.g., the nose and/or mouth), such as air, nitrogen, oxygen, any other component of air, CO₂, vaporized water, vaporized medicines, and/or any combination of two or more of the above, for example. As used herein, the term “patient” may refer to any person or animal that may receive breathing assistance from a breathing assistance system, regardless of the medical status, official patient status, physical location, or any other characteristic of the person. Thus, for example, patients may include persons under official medical care (e.g., hospital patients), persons not under official medical care, persons receiving care at a medical care facility, persons receiving home care, etc.

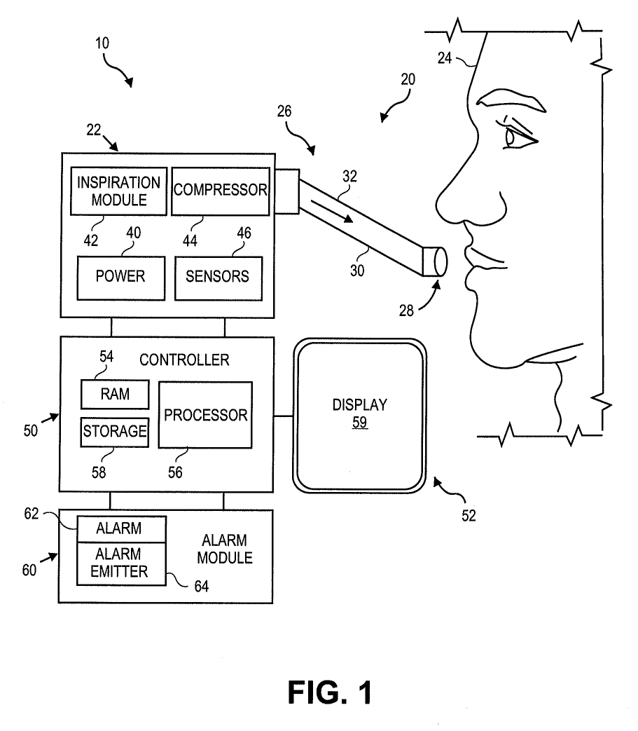

FIG. 1 illustrates an example breathing assistance system 10 according to certain embodiments of the present disclosure. Breathing assistance system 10 includes a ventilator 20 connected to a patient 24 by an airway 26. Ventilator 20 includes a pneumatic system 22, a controller 50, a display 59, and an alarm module 60. Pneumatic system 22 delivers breathing gases to and/or from patient 24 via airway 26, which couples patient 24 to pneumatic system 22 via physical patient interface 28 and breathing circuit 30. Breathing circuit 30 may include any conduits for communicating gas to and/or from patient 24, e.g., a one-limb or two-limb circuit for carrying gas to and/or from patient 24. The MPV or on-demand ventilation control systems described herein are generally used with an interface 28 to which a patient may selectively engage or disengage, as needed. One such type of interface 28 includes a rigid, semi-rigid, or flexible tube to which patients may selectively engage or disengage with the mouth as needed for inhalation and/or exhalation. Regardless, other non-invasive patient interfaces that may be utilized with the present technology include nasal masks, nasal/oral masks, full-face masks, nasal prongs, etc. A power module 40 may control the delivery of power to the various components, which may be delivered by a wall outlet or stand-alone power source (i.e., a battery). Pneumatic system 22 may also include a variety of other components, e.g., sources for pressurized air and/or oxygen, mixing modules, valves, tubing, accumulators, filters, etc., in addition to the components described in more detail below.

Pneumatic system 22 may be configured to receive gas from one or more sources, utilizing a number of different components. In the illustrated example, pneumatic system 22 includes an inspiration module 42 coupled with an inspiratory limb 32. In alternative embodiments, the pneumatic system 22 may include an expiration module coupled with an expiratory limb. In such a case, a two-limb circuit may be utilized for connection of the breathing circuit to the patient. A gas flow source 44 (e.g., a compressor, a turbine, one or more tanks of compressed gas, a wall outlet through which pressurized air may be supplied (e.g., in a hospital or clinic), etc.) is coupled with inspiration module 42 to provide a gas source for ventilatory support via inspiratory limb 32. In one embodiment, the gas flow source 44 is a turbine of low inertia and high rate speed. This turbine may be preceded by a filter for ambient air inlet and by an upstream and downstream sound deadening device. The turbine may have a maximum speed of rotation of about 50,000 rpm, adapted to supply a pressure of 70 millibar above ambient and a flow rate of about 200 l/min. The turbine is driven by an electric motor, controlled by the controller 50 so as to provide a wide range of flow rates and pressures.

Pneumatic system 22 also includes one or more sensors 46 for measuring various parameters or conditions. These sensors are described in more detail below. Although sensors 46 are illustrated as being located within pneumatic system 22, sensors 46 may be located at any suitable location or locations in breathing assistance system 10, e.g., within a housing of pneumatic system 22, along breathing circuit 30, coupled to patient interface 28, etc. The sensors 46 typically include devices that detect various conditions of the breathing assistance system 10. Various sensors, including pressure sensors, volume sensors, flow rate sensors, etc., may be utilized in the device. These sensors send signals to the controller, which in turn calculates pressure, flow rate, respiration rate, and other parameters, based on algorithms, look-up tables, or comparisons with outputs of other sensors. For example, a flow rate sensor may send a signal to the controller that, in turn, converts this signal to volume, pressure, or other readings based on the signal itself and other information. Regardless of the types of sensors utilized, many critical system conditions are regularly monitored to confirm proper operation of the breathing assistance system and to confirm that the patient is being ventilated as required. Such critical system conditions include those related to gas pressure, gas volume, patient respiration rate, gas minute volume, breathing circuit/ventilator connection, and patient apnea. In various standard operational modes, each of the above listed conditions may have high and low values, which may be set by an operator or patient, or that may be set automatically as part of selecting a particular operational mode. Values outside of the predetermined parameter range (defined by these high and low values) will cause a triggering event that will set off an alarm. Thereafter, the operator or patient may be required to take corrective action to silence the alarm.

Controller 50 is operatively coupled to pneumatic system 22 and an operator interface 52 enabling an operator (e.g., a physician or a patient) to interact with ventilator 20 (e.g., to change ventilator settings, select operational modes, view monitored parameters, etc.). The data storage 58 may include non-transitory, computer-readable storage media that stores software that is executed by the processor 56 and which controls the operation of the breathing assistance system 10. In an embodiment, the data storage 58 includes one or more solid-state storage devices such as flash memory chips. In an alternative embodiment, the data storage 58 may be mass storage connected to the processor 56 through a mass storage controller and a communications bus. Although the description of computer-readable media contained herein refers to a solid-state storage, it should be appreciated by those skilled in the art that computer-readable storage media can be any available media that can be accessed by the processor 56. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the breathing assistance system 10 or between the breathing assistance system 10 and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intranets or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination,

The controller 50 issues conunands to pneumatic system 22 in order to control the breathing assistance provided to patient 24 by ventilator 20. The specific commands may be based on inputs received from patient 24, pneumatic system 22 (e.g., from the pressure sensor, flow sensor, disconnect sensor, etc.), operator interface 52, and/or other components of breathing assistance system 10. In the illustrated example, operator interface 52 includes a display device 59. In some embodiments, display device 59 includes a touch-sensitive interface or other input devices, enabling display device 59 to serve both as an input and output device.

The alarm module 60 includes an alarm 62 and an alarm emitter 64 that signals activation of the alarm 62. The alarm emitter 64 may emit any or all of a visual, audible, or tactile signal to indicate to the patient or operator that a triggering event (as described in more detail) has occurred. Although the alarm module 60 is depicted as a discrete component, the alarm module may be incorporated into the controller 50, or one or more alarm modules may be directly connected to one or more of the sensors in the pneumatic system. In certain embodiments, different alarm emitters may be used to indicate which sensor has been activated. In general, although the pneumatic system 22, the controller 50, the operator interface 52, and the alarm module 60 are depicted as discrete components, any combination of components may be utilized (the alarm module may be incorporated into the controller, for example).

As described above, not all patients require assistance at all times for breathing. Certain patients, such as quadriplegic patients, may need a breathing assistance system to deliver gas on an as-needed basis, based on their own desired respiration rates. In that regard, typical breathing assistance systems may interpret this unusual respiration rate as a triggering event, such as apnea, requiring an alarm, In another example, a typical breathing assistance system would interpret the pressure conditions associated with a patient disengaging with the interface as a disconnect condition. This would also be considered a triggering event causing the breathing assistance system to energize the disconnect alarm. Under existing breathing assistance system operational modes, patients are compelled to adjust individual operational parameters of a breathing assistance system in an effort to avoid triggering various alarms. Still, even with resetting individual operational parameters, many of these parameters can not be adjusted or disabled so as to allow a patient to use a breathing assistance system at will and avoid all nuisance alarms. In that case, patients have had to tolerate alarms from breathing assistance systems that misinterpret the signals associated with MPV or on-demand ventilation.

Accordingly, an operational mode that does not allow activation of alarms based on triggering events that would otherwise trigger alarms in another mode is desirable to ensure the quality of life of the patients using a breathing assistance system. The proposed technology includes an operational mode, called an MPV or on-demand mode, where all critical alarms that would otherwise activate during other modes are not activated. Various controls and settings, including those described below, may be utilized to prevent activation of an alarm emitter in this mode.

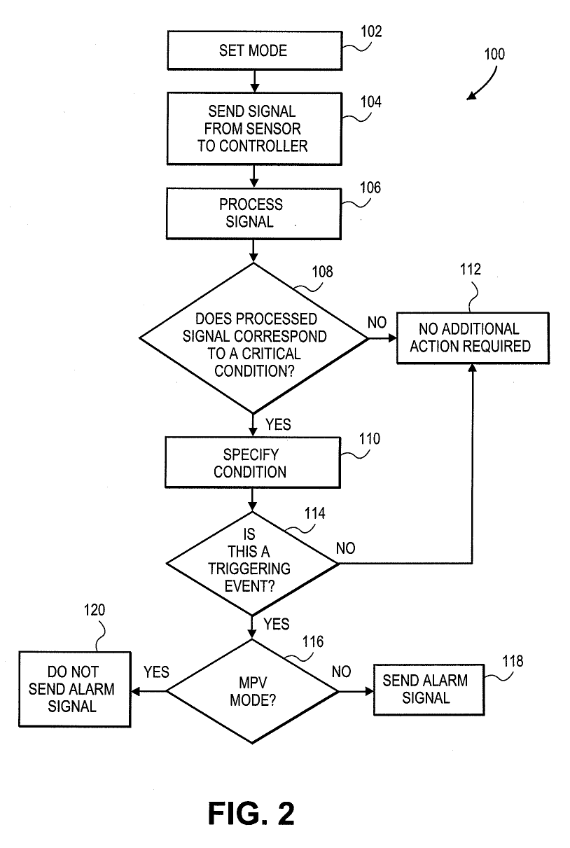

FIG. 2 depicts a method 100 of identifying and processing triggering events in a breathing assistance system. The method 100 includes setting an operational mode 102 of the system. A number of operational modes may be selected, depending on the particular functionality desired, patient requirements, etc. Typically, the mode is set by an operator or patient, and has configured therein a number of predefined parameters. In some embodiments, certain of these parameters may be adjusted by the operator or patient. During operation of the device, signals are sent from the sensor(s) to the controller 104. These signals may be binary or analog signals, corresponding to particular conditions within the system, as described above. The controller then processes the signals 106 and determines whether the processed signal corresponds to a critical condition 108. A critical condition could include, for example, a signal that is outside of a predetermined range for a signal from a particular sensor. Other critical conditions include those related to low pressure, low volume, low respiration rate, low minute volume, ventilator/breathing circuit disconnection, and apnea. If the signal does correspond to a critical condition, that condition is specified and relevant information is stored for future record-keeping purposes. If the signal does not correspond to a critical condition, no additional action is required 112 with regard to identifying and processing triggering events.

If a signal associated with a critical condition has been identified, the method 100 next determines whether a triggering event has occurred 114. If not, again, no additional action is required 112. If the processed signal does correspond to a triggering event, however, the system next determines whether the MPV mode has been set 116. If not, the controller sends an alarm signal to the alarm module 118 and an alarm is emitted (in other words, the system proceeds as it would in a normal operational mode). If the system is, in fact, in the MPV mode, no alarm signal will be sent to the emitter 120.

Modifications of this method that achieve the desired result of not activating alarms in response to triggering events in other modes are also contemplated. For example, the triggering event may be an actual, unprocessed signal sent by a particular sensor to a controller, in the case of a signal indicating a disconnect condition, for example. In another embodiment, the triggering event may be the absence of a signal sent to the controller. For example, proper connection of the breathing circuit and the ventilator may result in a constant signal being sent from the disconnect sensor, and the absence of that signal may indicate a disconnect condition, and a triggering event. In another embodiment, the alarm signal may attempt to send an alarm signal in MPV mode, but the power module 40 may not energize the alarm module 60, thus, no alarm signal will be emitted. In another embodiment, the power module 40 may not power the sensors 46 associated with certain critical conditions, or the controller 50 may be programmed to ignore any signals sent from such sensors 46 while in MPV mode.

In other examples of the MPV mode, the controller 50 will not send an alarm signal to the alarm module 60 when a signal from a sensor 46 is outside a predetermined range (in the case of a low volume or low pressure signal, for example). While this would ordinarily constitute a triggering event in a first mode, the controller 50 will not send an alarm signal to the alarm module 60 under this circumstance while in the MPV mode. In another embodiment of an MPV mode, a sensor 46 may be configured such that it will not send a signal to the controller 50 while in MPV mode. In that case, in the MPV mode, the controller 50 will not receive any signal and accordingly, can not send an alarm signal to the alarm module 60. In addition to the configurations identified above, other configurations that will prevent activation of alarms in MPV mode are also contemplated.

As described above, operators or patients wishing to use existing breathing assistance systems on an as-needed basis often have to adjust, reset, or otherwise configure the breathing assistance system so as to reduce the nuisance alarms that would be emitted under certain conditions. With the present technology, the breathing assistance system may be programmed either during manufacturing, or thereafter, to include an MPV mode. Programming the breathing assistance system after manufacturing may include installing software thereon, either via a network, flash drive, or other data storage medium. Programming the breathing assistance system may include introducing all the required settings so as to eliminate the energizing of alarms in the MPV mode. Additionally, one or more icons may be included on the display such that the MPV mode may be selected easily, and all conditions and settings required to eliminate nuisance alarms may be configured at once.

FIG, 3A depicts a pressure waveform for a prior art breathing assistance system, wherein a breath is delivered in response to a negative pressure trigger. In the depicted waveform, a bias flow pressure slightly elevated over ambient is constantly delivered in the absence of any further need by a patient. This bias airflow may be about 3 liters/minute or some other minimal airflow. At the beginning of patient effort, a drawing in of air through the breathing interface decreases airway pressure (that is, pressure within the breathing circuit and connected components of the breathing assistance system). The ventilator detects this trigger and begins delivering air almost immediately thereafter. The time between the beginning of patient effort and the delivery of breathing air may be defined by the control system, or otherwise set by a patient or operator. When the patient ceases effort (i.e., stops breathing in) the ventilator quickly returns to its initial bias flow state, until patient effort begins again.

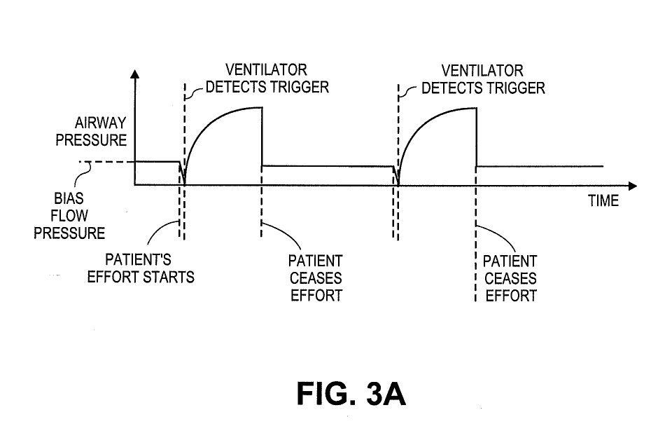

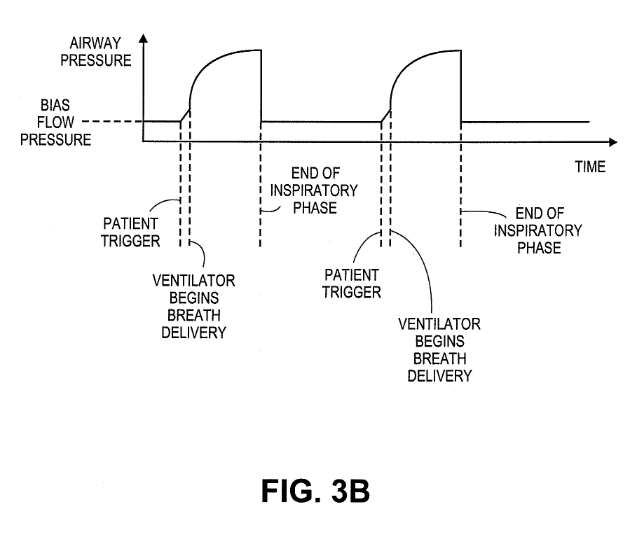

FIG. 3B depicts a pressure waveform for a breathing assistance system, wherein a breath is delivered in response to a positive pressure trigger, as described herein. In addition to conventional negative pressure triggering, the disclosed technology provides the very beneficial option of a positive pressure trigger to initiate the inspiratory phase. When no breath is required by the patient, the mouth is removed from the mouthpiece so there are no positive or negative pressure fluctuations. Alternatively, as in the depicted waveform, a small bias flow such as that provided by the prior art breathing assistance system is maintained. As soon as the patient latches onto the mouthpiece, this condition causes an increase in pressure within the airway. Depending on the sensitivity of the sensors, a pressure increase may even be detected in the absence of the bias flow, as the action of closing the mouth of the patient forces a small amount of air into the system. In another embodiment, the patient may trigger a breath by forcibly blowing in to the interface. Thereafter, the ventilator begins delivery of the breath and continues until the patient ceases effort (which could be unlatching from the patient interface). Alternatively, the inspiratory phase may terminate based on a predetermined time setting on the ventilator. In either case, at the end of the inspiratory phase, the ventilator quickly returns to its initial bias flow state, until patient effort begins again. In addition to the embodiments that utilize detection of pressure changes, directional sensors may be used to detect increases in airflow into the system (again corresponding to a patient latching on to or breathing in to the mouthpiece).

The inspiration module 42 may be configured to synchronize ventilation with a spontaneously-breathing, or triggering, patient who requires additional assistance. That is, the ventilator may be configured to detect patient effort, and may initiate gas delivery in response. Ventilation systems, depending on their breath type, may trigger and/or cycle automatically, or in response to a detection of patient effort, or both, Patient effort may be the result of a patient beginning to take a breath, which is generally depicted in FIG. 3B, In the MPV mode, however, patient effort may include any action that corresponds to the positive-pressure trigger described above. This action may include latching the mouth or closing the lips around the patient interface, or blowing into the patient interface.

The ventilator may detect patient effort via a pressure-monitoring method, a flow-monitoring method, or any other suitable method, Sensors may be either internal to the ventilator or breathing circuit and may include any suitable sensors. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight rise in circuit pressure, due to patient latching or blowing. Under positive-pressure triggering, the ventilator interprets the slight rise in circuit pressure as patient effort and consequently initiates inspiration by delivering respiratory gases. It should be noted that circuit pressure increases typically indicate obstruction within the breathing circuit. Thus, a breathing assistance system that operates on positive-pressure triggering would have to disable or otherwise ignore any pressure rises within the breathing circuit. This critical condition would therefore be ignored or otherwise not acted upon with alarms, as described above with regard to FIG. 2.

Alternatively, the ventilator may detect a flow-triggered event. Specifically, the ventilator may monitor the circuit flow. If the ventilator detects a slight drop in flow during bias flow, this may indicate that the patient is blowing into the interface, or has closed their lips around the interface. If bias flow is not being utilized, a flow within the circuit that was not initiated by the ventilator itself may also indicate that the patient is beginning patient effort (again, into the patient interface), and may initiate inspiration by delivering respiratory gases.

While there have been described herein what are to be considered exemplary and preferred embodiments of the present technology, other modifications of the technology will become apparent to those skilled in the art from the teachings herein. The particular methods of manufacture and geometries disclosed herein are exemplary in nature and are not to be considered limiting. It is therefore desired to be secured in the appended claims all such modifications as fall within the spirit and scope of the technology. Accordingly, what is desired to be secured by Letters Patent is the technology as defined and differentiated in the following claims, and all equivalents.

1. A ventilation system comprising:
a controller comprising a first mode setting and a second mode setting;
an alarm module in communication with the controller, the alarm module comprising an alarm and an alarm emitter indicating activation of the alarm, wherein the alarm is selected from the group consisting of a low pressure alarm, a low volume alarm, a low respiration rate alarm, a low minute volume alarm, a disconnect alarm, and an apnea alarm;
wherein when the controller is in the first mode setting, the alarm emitter is activated in response to a triggering event, and wherein when the controller is in the second mode setting, the alarm emitter is not activated in response to the triggering event.

2. The ventilation system of claim 1,
wherein when the controller is in the first mode setting, the controller sends an alarm signal to the alarm module based on the triggering event, wherein the alarm signal activates the alarm emitter, and
wherein when the controller is in the second mode setting, the controller does not send the alarm signal to the alarm module based on the triggering event.

3. The ventilation system of claim 2, further comprising a sensor in communication with the controller, wherein the triggering event comprises a signal sent by the sensor to the controller.

4. The ventilation system of claim 3, triggering event comprises a signal having a value outside of a predetermined range.

5. The ventilation system of claim 1,
wherein when the controller is in the first mode setting, the controller sends an alarm signal to the alarm module based on the triggering event, wherein the alarm signal activates the alarm emitter, and
wherein when the controller is in the second mode setting, the controller does not send the alarm signal to the alarm module based on the absence of the triggering event.

6. The ventilation system of claim 5, further comprising a sensor in communication with the controller, wherein the triggering event comprises a signal sent by the sensor to the controller.

7. The ventilation system of claim 1, further comprising a sensor in communication with the controller,
wherein when the controller is in the first mode setting, the sensor sends a signal to the controller, and
wherein when the controller is in the second mode setting, the sensor does not send the signal to the controller, and
wherein the controller activates the alarm emitter based on receipt of the signal.

8. The ventilation system of claim 1, further comprising a power module in communication with the controller,
wherein when the controller is in the first mode setting, the power module energizes the alarm emitter, and
wherein when the controller is in the second mode setting, the power module does not energize the alarm emitter.

9. A method of controlling an alarm state in a ventilation system comprising a controller, a power module, a sensor module, and an alarm emitter activated by at least one of a low pressure signal and a low volume signal, the method comprising the step of
programming the ventilation system to comprise an on-demand ventilation mode, wherein in the on-demand ventilation mode, the alarm emitter is not activated.

10. The method of claim 9, wherein when in the on-demand ventilation mode, the controller does not send an alarm signal to the alarm emitter.

11. The method of claim 9, wherein when in the on-demand ventilation mode, the power module does not deliver power to the alarm emitter.

12. The method of claim 9, wherein when in the on-demand ventilation mode, the sensor module does not send a signal to the controller.

13. The method of claim 9, wherein when in the on-demand ventilation mode, the sensor module sends a signal to the controller, wherein the signal does not meet a predetermined threshold value.

14. The method of claim 9, wherein when in the on-demand ventilation mode, the power module does not deliver power to the sensor module.

15. A ventilation system comprising:
a breathing circuit;
an airflow generator for delivering a ventilation airflow to the breathing circuit; and
a sensor for sensing at least one of an air flow or air pressure within the breathing circuit, wherein the ventilation airflow is delivered to the breathing circuit upon an increase in at least one of the air flow and the air pressure.

16. The ventilation system of claim 15, further comprising a patient interface defining an opening, wherein the patient interface is connected to the breathing circuit.

17. The ventilation system of claim 16, wherein the increase at least one of the air flow and the air pressure is caused by a patient forcing air into the interface.

18. The ventilation system of claim 16, wherein the increase at least one of the air flow and the air pressure is caused by a patient orally latching on to the interface,

19. The ventilation system of claim 16, wherein the increase at least one of the air flow and the air pressure is caused by a patient at least partially blocking the opening.

20. The ventilation system of claim 16, wherein the increase at least one of the air flow and the air pressure is caused by a patient at least partially obstructing a biasing airflow through the opening.
